(19) United States
(12) Patent Application Publication
(10) Pub. No.: US 2012/0172891 A1
(43) Pub. Date: Jul. 5, 2012

(54) IMPLANTABLE MEDICAL DEVICE FIXATION TESTING

(75) Inventor: Nathan T. Lee, Golden Valley, MN (US)

(73) Assignee: Medtronic, Inc., Minneapolis, MN (US)

(21) Appl. No.: 13/074,948

(22) Filed: Mar. 29, 2011

Related U.S. Application Data
(60) Provisional application No. 61/428,127, filed on Dec. 29, 2010.

(51) Int. Cl. A61B 19/00

(52) U.S. Cl. 606/129

(57) ABSTRACT

In one example, this disclosure includes a kit for implanting an implantable medical device within a patient. The kit comprises a delivery catheter including an inner member and an outer member. The kit further comprises the implantable medical device. The implantable medical device is adjacent the inner member and constrained by the outer member. The kit further comprises a force sensor in mechanical communication with the implantable medical device via the inner member. The force sensor collects force feedback data representing force applied by the inner member on the implantable medical device. The kit further comprises a user communication module configured to deliver force feedback information corresponding to the force feedback data collected by the force sensor to a user.

### TECHNICAL FIELD

This disclosure relates to fixation techniques for implantable medical devices.

### BACKGROUND

Medical devices such as electrical stimulators, leads, and electrodes are implanted to deliver therapy to one or more target sites within the body of a patient. To ensure reliable electrical contact between the electrodes and the target site, fixation of the device, lead, or electrodes is desirable.

A variety of medical devices for delivering a therapy and/or monitoring physiological conditions have been used clinically or proposed for clinical use in patients. Examples include medical devices that deliver therapy to and/or monitor conditions associated with the heart, muscle, nerve, brain, stomach or other organs or tissue. Some therapies include the delivery of electrical signals, e.g., stimulation, to such organs or tissues. Some medical devices may employ one or more elongated electrical leads carrying electrodes for the delivery of therapeutic electrical signals to such organs or tissues, electrodes for sensing intrinsic electrical signals within the patient, which may be generated by such organs or tissue, and/or other sensors for sensing physiological parameters of a patient.

Medical leads may be configured to allow electrodes or other sensors to be positioned at desired locations for delivery of therapeutic electrical signals or sensing. For example, electrodes or sensors may be carried at a distal portion of a lead. A proximal portion of the lead may be coupled to a medical device housing, which may contain circuitry such as signal generation and/or sensing circuitry. In some cases, the medical leads and the medical device housing are implantable within the patient. Medical devices with a housing configured for implantation within the patient may be referred to as implantable medical devices (IMDs).

Implantable cardiac pacemakers or cardioverter-defibrillators, for example, provide therapeutic electrical signals to the heart, e.g., via electrodes carried by one or more implantable medical leads. The therapeutic electrical signals may include pulses for pacing, or shocks for cardioversion or defibrillation. In some cases, a medical device may sense intrinsic depolarizations of the heart, and control delivery of therapeutic signals to the heart based on the sensed depolarizations. Upon detection of an abnormal rhythm, such as bradycardia, tachycardia or fibrillation, an appropriate therapeutic electrical signal or signals may be delivered to restore or maintain a more normal rhythm. For example, in some cases, an IMD may deliver pacing stimulation to the heart of the patient upon detecting tachycardia or bradycardia, and deliver cardioversion or defibrillation shocks to the heart upon detecting fibrillation.

Leadless IMDs may also be used to deliver therapy to a patient, and/or sense physiological parameters of a patient. In some examples, a leadless IMD may include one or more electrodes on its outer housing to deliver therapeutic electrical signals to patient, and/or sense intrinsic electrical signals of patient. For example, leadless cardiac devices, such as leadless pacemakers, may also be used to sense intrinsic depolarizations and/or other physiological parameters of the heart and/or deliver therapeutic electrical signals to the heart. A leadless cardiac device may include one or more electrodes on its outer housing to deliver therapeutic electrical signals and/or sense intrinsic depolarizations of the heart. Leadless cardiac devices may be positioned within or outside of the heart and, in some examples, may be anchored to a wall of the heart via a fixation mechanism.

### SUMMARY

In general, this disclosure describes techniques for verifying adequate fixation of IMDs implanted within a patient. As an example, a delivery device, such as a delivery catheter, may include a force sensor that can provide a representation of a holding force of an IMD. Alternatively or in addition to providing a representation of a holding force of an IMD, a force sensor may provide a representation of a deployment force applied by the catheter on the IMD. The catheter may further include a user communication module that delivers force feedback information to a user. The user may evaluate the force feedback information to determine if the holding force of the IMD is adequate before fully releasing the IMD from the catheter.

In one example, the disclosure is directed to a kit for implanting an implantable medical device within a patient. The kit comprises a delivery catheter including an inner member and an outer member. The kit further comprises the implantable medical device. The implantable medical device is adjacent the inner member and constrained by the outer member. The kit further comprises a force sensor in mechanical communication with the implantable medical device via the inner member. The force sensor collects force feedback data representing force applied by the inner member on the implantable medical device. The kit further comprises a user communication module configured to deliver force feedback information corresponding to the force feedback data collected by the force sensor to a user.

In another example, the disclosure is directed to a catheter for implanting an implantable medical device within a patient, the catheter comprising: an inner member configured to apply a force to the implantable medical device, an outer member configured to constrain the implantable medical device, and a force sensor configured to collect force feedback data representing force applied by the inner member on the implantable medical device; and a user communication module configured to deliver force feedback information corresponding to the force feedback data collected by the force sensor to a user.

In another example, the disclosure is directed to a method of implanting an implantable medical device within a patient comprising: deploying the implantable medical device from a catheter to a location within the patient, the catheter including a force sensor in mechanical communication with the implantable medical device; receiving an indication of a holding force of the implantable medical device, wherein the indication of the holding force corresponds to force feedback data collected by the force sensor; and fully releasing the implantable medical device from the catheter at the location within the patient after determining the implantable medical device is adequately fixated at the location within the patient. Determining the implantable medical device is adequately fixated at the location within the patient comprises evaluating whether the implantable medical device is adequately fixated at the location within the patient based on the indication of the holding force of the implantable medical device.

The details of one or more aspects of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a conceptual diagram illustrating an example therapy system comprising a leadless IMD that may be used to monitor one or more physiological parameters of a patient and/or provide therapy to the heart of a patient.

FIG. 2 is a conceptual diagram illustrating another example therapy system comprising a leadless IMD that may be used to monitor one or more physiological parameters of a patient and/or provide therapy to the heart of a patient.

FIG. 3 illustrates the leadless IMD of FIG. 1 in further detail.

FIG. 4 illustrates an assembly including the leadless IMD of FIG. 1 and a catheter configured to deploy the leadless IMD of FIG. 1.

FIG. 5 illustrates the leadless IMD of FIG. 2 in further detail.

FIG. 6 illustrates an assembly including the leadless IMD of FIG. 2 and a catheter configured to deploy the leadless IMD of FIG. 2.

FIG. 7 is a functional block diagram illustrating an example configuration of the IMD of FIG. 1.

FIG. 8 is a functional block diagram illustrating an example configuration of the IMD of FIG. 2.

FIG. 9 is a block diagram of an example external programmer that facilitates user communication with an IMD.

FIG. 10 is a flowchart illustrating techniques for implanting an implantable medical device within a patient.

### DETAILED DESCRIPTION

Minimally invasive surgery, such as percutaneous surgery, permits IMD implantation with less pain and recovery time than open surgery. However, minimally invasive surgery tends to be more complicated than open surgery. For example, fixating a device may require a surgeon to manipulate instruments remotely, e.g., within the confines of an intravascular catheter. With techniques for remote deployment and fixation of IMDs, it can be difficult to ensure adequate fixation. As one example, ensuring adequate fixation of leadless implantable medical devices (IMDs) during an implantation procedure can be particularly difficult as a clinician does not have direct access to the IMD following fixation. While fluoroscopy may be used to verify whether an leadless IMD is fully deployed from a delivery catheter and to verify the leadless IMD is in a stable position, fluoroscopy is not suitable for evaluating whether the IMD is adequately fixated, e.g., fixated with a holding force associated with an acceptably low risk of future migration or dislodgement of the IMD.

This disclosure includes techniques for verifying adequate fixation of IMDs implanted within a patient. For example, a catheter may include a force sensor that can provide a representation of a holding force of an IMD. The catheter may include a user communication module that delivers force feedback information corresponding to the force feedback data collected by the force sensor to a user. The user may evaluate the force feedback information to determine if the holding force of the IMD is adequate before fully releasing the IMD from the catheter.

Although various examples are described with respect to leadless pacemakers and leadless IMDs deployed in the pulmonary artery, the techniques may be useful to verify fixation during implantation of a variety of implantable medical devices in a variety of anatomical locations. For example, the described techniques can be readily applied to verify fixation during implantation of any IMD located within a vessel, including leadless IMDs comprising sensors such as, but not limited to, a pressure sensor, an electrocardiogram sensor, a fluid flow sensor, a tissue oxygen sensor, an accelerometer, a glucose sensor, a potassium sensor, a thermometer and/or other sensors.

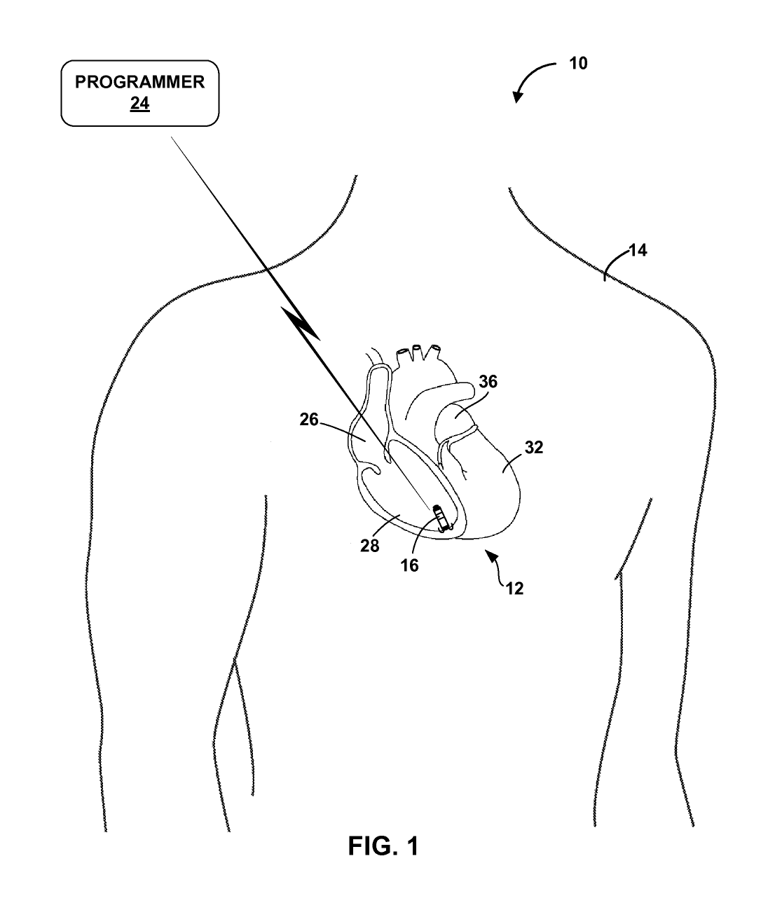

FIG. 1 is a conceptual diagram illustrating an example therapy system 10 that may be used to monitor one or more physiological parameters of patient 14 and/or to provide therapy to heart 12 of patient 14. Therapy system 10 includes IMD 16, which is coupled to programmer 24. IMD 16 may be an implantable leadless pacemaker that provides electrical signals to heart 12 via one or more electrodes (not shown in FIG. 1) on its outer housing. Additionally or alternatively, IMD 16 may sense electrical signals attendant to the depolarization and repolarization of heart 12 via electrodes on its outer housing. In some examples, IMD 16 provides pacing pulses to heart 12 based on the electrical signals sensed within heart 12.

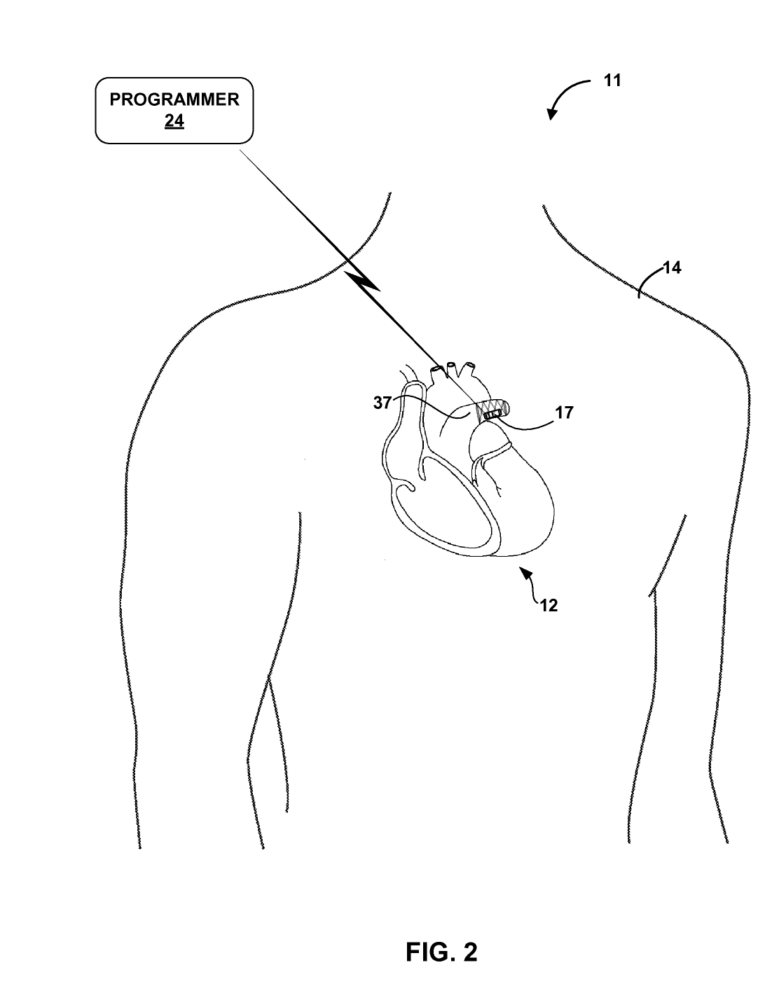

IMD 16 includes a set of active fixation tines to secure IMD 16 to a patient tissue. In other examples, IMD 16 may be secured with other techniques such as a helical screw or with an expandable fixation element (as described with respect to IMD 17 of FIG. 2). In the example of FIG. 1, IMD 16 is positioned wholly within heart 12 proximate to an inner wall of right ventricle 28 to provide right ventricular (RV) pacing. Although IMD 16 is shown within heart 12 and proximate to an inner wall of right ventricle 28 in the example of FIG. 1, IMD 16 may be positioned at any other location outside or within heart 12. For example, IMD 16 may be positioned outside or within right atrium 26, left atrium 36, and/or left ventricle 32, e.g., to provide right atrial, left atrial, and left ventricular pacing, respectively.

Depending on the location of implant, IMD 16 may include other stimulation functionalities. For example, IMD 16 may provide atrioventricular nodal stimulation, fat pad stimulation, vagal stimulation, or other types of neurostimulation. In other examples, IMD 16 may be a monitor that senses one or more parameters of heart 12 and may not provide any stimulation functionality. In some examples, therapy system 10 may include a plurality of leadless IMDs 16, e.g., to provide stimulation and/or sensing at a variety of locations.

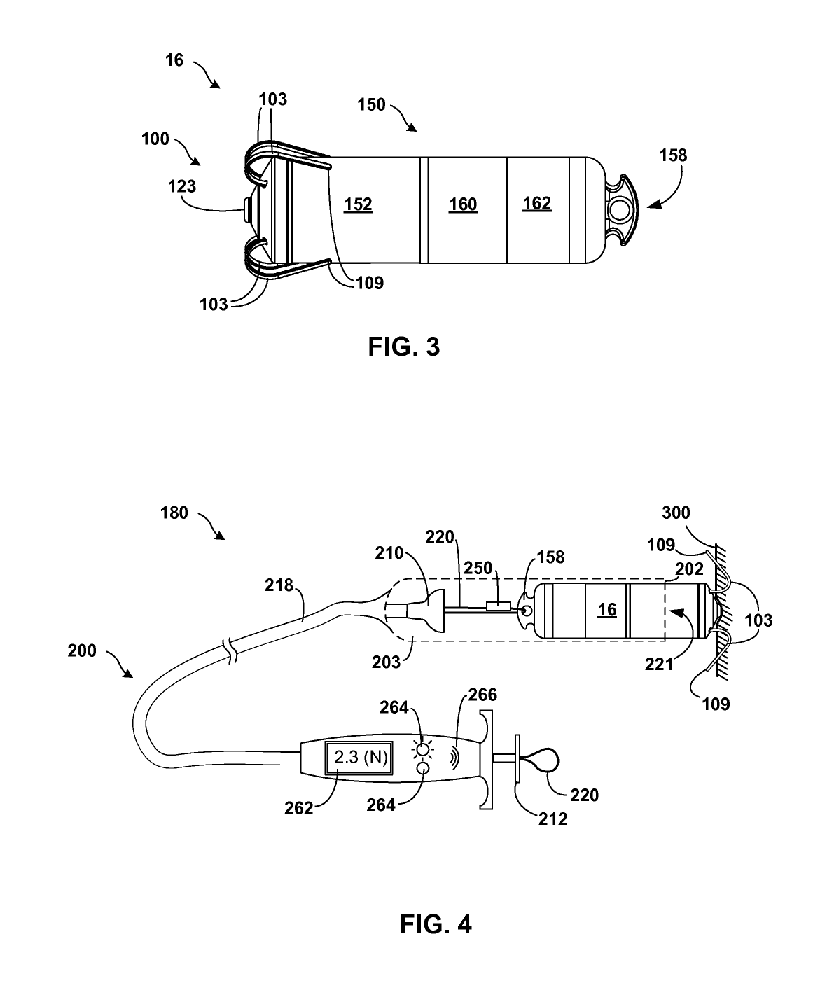

As discussed in greater detail with respect to FIG. 3, IMD 16 includes a set of active fixation tines. The active fixation tines in the set are deployable from a spring-loaded position in which distal ends of the active fixation tines point away from the IMD to a hooked position in which the active fixation tines bend back towards the IMD. The active fixation tines allow IMD 16 to be removed from a patient tissue followed by redeployment, e.g., to adjust the position of IMD 16 relative to the patient tissue. For example, a clinician implanting IMD 16 may reposition IMD 16 during an implantation procedure if the original deployment of the active fixation tines provides an insufficient holding force to reliably secure IMD 16 to the patient tissue. As another example, the clinician may reposition IMD 16 during an implantation procedure if testing of IMD 16 indicates an unacceptably high capture threshold, which may be caused by, e.g., the specific location of IMD 16 or a poor electrode-tissue connection.

For example, as discussed in greater detail with respect to FIG. 4, the clinician may implant IMD 16 using a catheter including a force sensor that can provide a representation of a holding force of IMD 16 after deployment. The catheter may include a user communication module that delivers force feedback information collected by the force sensor to the clinician. Based on the force feedback information, the clinician can determine if the holding force of IMD 16 is adequate before fully releasing IMD 16 from the catheter.

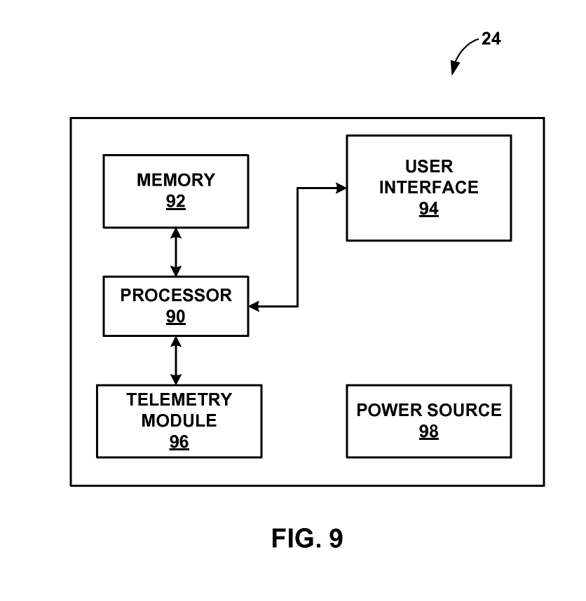

FIG. 1 further depicts programmer 24 in wireless communication with IMD 16. In some examples, programmer 24 comprises a handheld computing device, computer workstation, or networked computing device. Programmer 24, shown and described in more detail below with respect to FIG. 9, includes a user interface that presents information to and receives input from a user. It should be noted that the user may also interact with programmer 24 remotely via a networked computing device.

A user, such as a physician, technician, surgeon, electrophysiologist, other clinician, or patient, interacts with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD 16. For example, the user may use programmer 24 to retrieve information from IMD 16 regarding the rhythm of heart 12, trends therein over time, or arrhythmic episodes.

As an example, the user may use programmer 24 to retrieve information from IMD 16 regarding other sensed physiological parameters of patient 14 or information derived from sensed physiological parameters, such as intracardiac or intravascular pressure, intracardiac or intravascular fluid flow, activity, posture, tissue oxygen levels, respiration, tissue perfusion, heart sounds, cardiac electrogram (EGM), intracardiac impedance, or thoracic impedance. In some examples, the user may use programmer 24 to retrieve information from IMD 16 regarding the performance or integrity of IMD 16 or other components of system 16, or a power source of IMD 16. As another example, the user may interact with programmer 24 to program, e.g., select parameters for, therapies provided by IMD 16, such as pacing and, optionally, neurostimulation.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

FIG. 2 is a conceptual diagram illustrating an example therapy system 11 that may be used to monitor one or more physiological parameters of patient 14. System 11 includes IMD 17, which is coupled to programmer 24. IMD 17 may be an implantable leadless sensor that monitors one or more physiological conditions of patient 14 via one or more sensors (not shown in FIG. 1). As shown in FIG. 2, IMD 17 is located within a branch of pulmonary artery 37 of patient 14, such as the left or right pulmonary artery. As one example, IMD 17 may measure pressure within pulmonary artery 37. In other examples, IMD 17 may be implanted within other body lumens, such as other vasculature of patient 14. Additionally or alternatively to including a pressure sensor, IMD 17 may also include sensors such as, but not limited to an electrocardiogram sensor, a fluid flow sensor, a tissue oxygen sensor, an accelerometer, a glucose sensor, a potassium sensor, a thermometer and/or other sensors. In some examples, system 11 may include a plurality of leadless IMDs 17, e.g., to provide sensing of one or more physiological conditions of patient 14 at a variety of locations.

As discussed in greater detail with respect to FIG. 6, IMD 17 includes an expandable fixation element. The expandable fixation element is configured such that the outer diameter of the expandable fixation element is expandable to provide an interference fit with the inner diameter of pulmonary artery 37, or other body lumen. In some examples, as also discussed with respect to FIG. 6, the expandable fixation element may be partially deployable. As an example, the distal end of the expandable fixation element may be deployed from a catheter and expanded to provide an interference fit with the body lumen while the proximal end of the expandable fixation element may remain in a collapsed position within the distal end of the catheter.

The expandable fixation element allows IMD 17 to be retracted before fully deploying IMD 17, e.g., to adjust the position of IMD 17 with a vasculature to a location in the vasculature providing a tighter (or looser) interference fit. For example, a clinician implanting IMD 17 may reposition IMD 17 during an implantation procedure if partial deployment of the expandable fixation element provides an insufficient holding force indicating that full deployment of the expandable fixation element may not reliably secure IMD 17 within the vasculature. As another example, a clinician may select an expandable fixation element with a size better suited for the vasculature than the expandable fixation element that provided an insufficient holding force.

The clinician may implant IMD 17 using a catheter including a force sensor that can provide a representation of a holding force of IMD 17 after partial deployment. The catheter may include a user communication module that delivers force feedback information collected by the force sensor to the clinician. Based on the force feedback information, the clinician can to determine if the holding force of IMD 17 is adequate before fully releasing IMD 17 from the catheter.

FIG. 2 further depicts programmer 24 in wireless communication with IMD 17. As with IMD 16 of FIG. 1, programmer 24 may be used to communicate with IMD 17.

FIG. 3 illustrates leadless IMD 16 of FIG. 1 in further detail. In the example of FIG. 3, leadless IMD 16 includes tine fixation subassembly 100 and electronic subassembly 150. Tine fixation subassembly 100 includes active fixation tines 103 and is configured to deploy anchor leadless IMD 16 to a patient tissue, such as a wall of heart 12.

Electronic subassembly 150 includes control electronics 152, which controls the sensing and/or therapy functions of IMD 16, and battery 160, which powers control electronics 152. As one example, control electronics 152 may include sensing circuitry, a stimulation generator and a telemetry module. As one example, battery 160 may comprise features of the batteries disclosed in U.S. patent application Ser. No. 12/696,890, titled IMPLANTABLE MEDICAL DEVICE BATTERY and filed Jan. 29, 2010, the entire contents of which are incorporated by reference herein.

The housings of control electronics 152 and battery 160 are formed from a biocompatible material, such as a stainless steel or titanium alloy. In some examples, the housings of control electronics 152 and battery 160 may include a parylene coating. Electronic subassembly 150 further includes anode 162, which may include a titanium nitride coating. The entirety of the housings of control electronics 152 and battery 160 are electrically connected to one another, but only anode 162 is uninsulated. Alternatively, anode 162 may be electrically isolated from the other portions of the housings of control electronics 152 and battery 160. In other examples, the entirety of the housing of battery 160 or the entirety of the housing of electronic subassembly 150 may function as an anode instead of providing a localized anode such as anode 162.

Delivery tool interface 158 is located at the proximal end of electronic subassembly 150. Delivery tool interface 158 is configured to connect to a delivery device, such as catheter 200 (FIG. 4) used to position IMD 16 during an implantation procedure.

Active fixation tines 103 are deployable from a spring-loaded position in which distal ends 109 of active fixation tines 103 point away from electronic subassembly 150 to a hooked position in which active fixation tines 103 bend back towards electronic subassembly 150. For example, active fixation tines 103 are shown in a hooked position in FIG. 3. Active fixation tines 103 may be fabricated of a shape memory material, which allows active fixation tines 103 to bend elastically from the hooked position to the spring-loaded position. As an example, the shape memory material may be shape memory alloy such as Nitinol.

In some examples, all or a portion of tine fixation subassembly 100, such as active fixation tines 103, may include one or more coatings. For example, tine fixation subassembly 100 may include a radiopaque coating to provide visibility during fluoroscopy. In one such example, fixation element 102 may include one or more radiopaque markers. As another example, active fixation tines 103 may be coated with a tissue growth promoter or a tissue growth inhibitor. A tissue growth promoter may be useful to increase the holding force of active fixation tines 103, whereas a tissue growth inhibitor may be useful to facilitate removal of IMD 16 during an explantation procedure, which may occur many years after the implantation of IMD 16.

As one example, IMD 16 and active fixation tines 103 may comprise features of the active fixation tines disclosed in U.S. Provisional Pat. App. No. 61/428,067, titled, “IMPLANTABLE MEDICAL DEVICE FIXATION” and filed Dec. 29, 2010, the entire contents of which are incorporated by reference herein.

FIG. 4 illustrates assembly 180, which includes leadless IMD 16 and catheter 200, which is configured to deliver leadless IMD 16 to the right ventricle of the patient and remotely deploy IMD 16. As shown in FIG. 4, active fixation tines 103 of IMD 16 are deployed in patient tissue 300.

Catheter 200 may be a steerable catheter or be configured to traverse a guidewire. In any case, catheter 200 may be directed within a body lumen, such as a vascular structure, to a target site in order to facilitate remote positioning and deployment of IMD 16. Catheter 200 comprises outer member 218, deployment element 210 and tether 220. Deployment element 210 and tether 220 can each be more generally referred to as inner members of catheter 200. Outer member 218 forms lumen 203, which is sized to receive IMD 16 at distal end 202 of catheter 200. For example, the inner diameter of lumen 203 at the distal end of catheter 200 may be about the same size as the outer diameter of IMD 16. When IMD 16 is positioned within lumen 203 at the distal end of catheter 200, lumen 203 of outer member 218 constrains IMD 16 and holds active fixation tines 103 in a spring-loaded position. In the spring-loaded position, active fixation tines 103 store enough potential energy to secure IMD 16 to a patient tissue upon deployment.

Lumen 203 includes aperture 221, which is positioned at distal end 202 of catheter 200. Aperture 221 facilitates deployment of IMD 16. Deployment element 210 is positioned proximate to IMD 16 in lumen 203. Deployment element 210 is configured to initiate deployment of active fixation tines 103. More particularly, a clinician may remotely deploy IMD 16 by pressing plunger 212, which is located at the proximal end of catheter 200. Plunger 212 connects directly to deployment element 210, e.g., with a wire or other stiff element running through outer member 218, such that pressing on plunger 212 moves deployment element 210 distally within lumen 203. As deployment element 210 moves distally within lumen 203, deployment element 210 pushes IMD 16 distally within lumen 203 and towards aperture 221. Once distal ends 109 of active fixation tines 103 reach aperture 221, active fixation tines 103 pull IMD 16 out of lumen 203 via aperture 221 as active fixation tines 103 move from a spring-loaded position to a hooked position to deploy IMD 16. The potential energy released by active fixation tines 103 upon deployment is sufficient to penetrate a patient tissue and secure IMD 16 to the patient tissue.

Tether 220 is attached to delivery tool interface 158 of IMD 16 and extends through catheter 200. Following deployment of IMD 16, a clinician may remotely pull IMD 16 back into lumen 203 by pulling on tether 220 at the proximal end of catheter 200. Pulling IMD 16 back into lumen 203 returns active fixation tines 103 to the spring-loaded position from the hooked position. The proximal ends of active fixation tines 103 remain fixed to the housing of IMD 16 as active fixation tines 103 move from the spring-loaded position from the hooked position and vice-versa. In some examples, active fixation tines 103 are configured to facilitate releasing IMD 16 from patient tissue without tearing the tissue when IMD 16 is pulled back into lumen 203 by tether 220. A clinician may redeploy IMD 16 with deployment element 210 by again operating plunger 212.

Catheter 200 further includes force sensor 250, which is located on tether 220. Force sensor 250 is in mechanical communication with IMD 16 via tether 220. Force sensor 250 collects force feedback data representing force applied by tether 220 on IMD 16. For example, force sensor 250 collects force feedback data representing a pull force of tether 220 on IMD 16. Force sensor 250 is located near the distal end of tether 220 so that force measurements will not be significantly impacted by friction between outer member 218 and tether 220. In another example, catheter 200 could include a force sensor that collects force feedback information representing a pushing force of deployment element 210 on IMD 16 as a clinician user attempts to deploy IMD 16 from catheter 200. Such force information could indicate to a clinician a potential hang-up between IMD 16 and catheter 200, e.g., between active fixation tines 103 and an inner wall of outer member 218 or more importantly, excessive deployment force being applied on patient tissue during deployment, which could cause injury to the patient tissue. In such an instance, the clinician could pull tether 220 to recapture IMD 16, readjust positioning of catheter 200 and reattempt deployment.

In different examples, force sensor 250 may be a fiber optic strain sensor or an electronic strain gauge, such as a quarter bridge strain gauge. In one example, force sensor 250 may be a fiber optic strain sensor including techniques disclosed in U.S. Pat. Pub. No. 2010/0030063, titled, “SYSTEM AND METHOD FOR TRACKING AN INSTRUMENT” and dated Feb. 4, 2010, the entire contents of which are incorporated by reference herein. In addition, as of the filing date of this disclosure, electronic strain gauges suitable for use as force sensor 250 include Arthroscopically Implantable Force Probes available from MicroStrain, Inc. of Williston, Vt., United States of America, although other electronic strain gauges may also be used.

Force sensor 250 may be used by a clinician to determine if a holding force of IMD 16 at least meets a predetermined threshold level. To determine whether a holding force of IMD 16 at least meets a predetermined threshold level, a clinician first deploys active fixation tines 103 into patient tissue 300. Then the clinician pulls on tether 220 at the proximal end of catheter 200 while monitoring force feedback information corresponding to the force feedback data collected by force sensor 250. Once the force feedback information monitored by the clinician indicates that the holding force of IMD 16 at least meets a predetermined threshold level, the clinician may stop pulling on tether 220 to prevent dislodging IMD 16 from patient tissue 300. Alternatively, if the holding force of IMD 16 does not at least meet a predetermined threshold level, IMD 16 will dislodge from patient tissue 300 before the force feedback information indicates that the holding force of IMD 16 at least meets a predetermined threshold level. In such a circumstance, the clinician may recapture IMD 16 by pulling on tether 220 and redeploy IMD 16. Fluoroscope or other imaging or navigation technique can be used by physician at the same time the holding force of the IMD 16 is tested to aid in determining if IMD 16 has physically moved prior to holding force threshold level being met.

Catheter 200 includes a variety of exemplary user communication modules suitable for delivering force feedback information corresponding to the force feedback data collected by force sensor 250 to the clinician. In particular, catheter 200 includes digital readout 262, which provides real-time representation of the force feedback of force sensor 250, visible alert 264, which is depicted in FIG. 4 as two light-emitting-diodes (LEDs) and audible alert 266. In one example, digital readout 262 or another display, such as a remote display may provide a graphical user interface display of force versus time. Digital readout 262, visible alert 264 and audible alert 266 may each be more generally characterized as a user communication module configured to deliver force feedback information corresponding to the force feedback data collected by force sensor 250 to a user.

In one example, digital readout 262 provides a real time measurement of the force experienced by tether 220 on IMD 16. Because tether 220 is a loop and therefore includes two longitudinal segments, the actual force measured by force sensor 250 may be doubled prior to being displayed on digital readout 262 to provide an accurate representation of the force applied on IMD 16 by tether 220. In other examples, a tether or other inner member may include only one longitudinal segment, and the actual force measured may be displayed on digital readout 262. The force sensor 250 may perform measurement sampling at various frequencies such as between 50 to 200 Hz.

Visible alert 264 may provide force feedback information indicating whether force sensor 250 is measuring a force that at least meets a predetermined threshold level. For example, visible alert 264 may include a first LED (e.g., a green LED) that lights-up when the force measured by force sensor 250 meets or exceeds a predetermined threshold level holding force of IMD 16 and a second LED (e.g., a red LED) that lights-up when the force measured by force sensor 250 meets or exceeds a predetermined threshold indicating that additional force may be expected to result in dislodgement of IMD 16 from patient tissue 300, which would be a predetermined threshold level exceeding the predetermined threshold level of the first LED. For example, the second LED may be useful to help prevent a clinician from accidentally dislodging IMD 16 when testing the holding force of active fixation tines 103 in patient tissue 300.

As another example, audible alert 266 may be used in addition to or instead of one or both of digital readout 262 and visible alert 264. For example, audible alert 266 may provide an auditory signal indicating force sensor 250 is measuring a force that at least meets a predetermined threshold level. In addition, audible alert 266 may further provide one or more additional auditory signals indicating force sensor 250 is measuring a force that at least meets a higher predetermined threshold level. As one example, audible alert 266 may emit a series of beeps that get progressively faster and/or louder as the force measured by force sensor 250 increasingly exceeds a predetermined threshold level holding force of IMD 16. As with visible alert 264, audible alert 266 may be useful to help prevent a clinician from accidentally dislodging IMD 16 when testing the holding force of active fixation tines 103 in patient tissue 300. In other examples, a clinician may receive force feedback information corresponding to the force feedback data collected by force sensor 250 from a device, e.g., a device similar to programmer 24, that is in wireless communication with force sensor 250.

Based on the force feedback information collected by force sensor 250, the clinician can determine if the holding force of IMD 16 is adequate to provide acceptably low risks of future migration or dislodgement of 16 before fully releasing IMD 16 from catheter 200. Fully releasing IMD 16 from the catheter 200 includes releasing IMD 16 from tether 220 and withdrawing catheter 200 such that the entirety of IMD 16 exits aperture 221 at distal end 202 of catheter 200. For example, the clinician may sever tether 220 at the proximal end of catheter 200 and remove tether 220 from delivery tool interface 158 by pulling on one of the severed ends of tether 220.

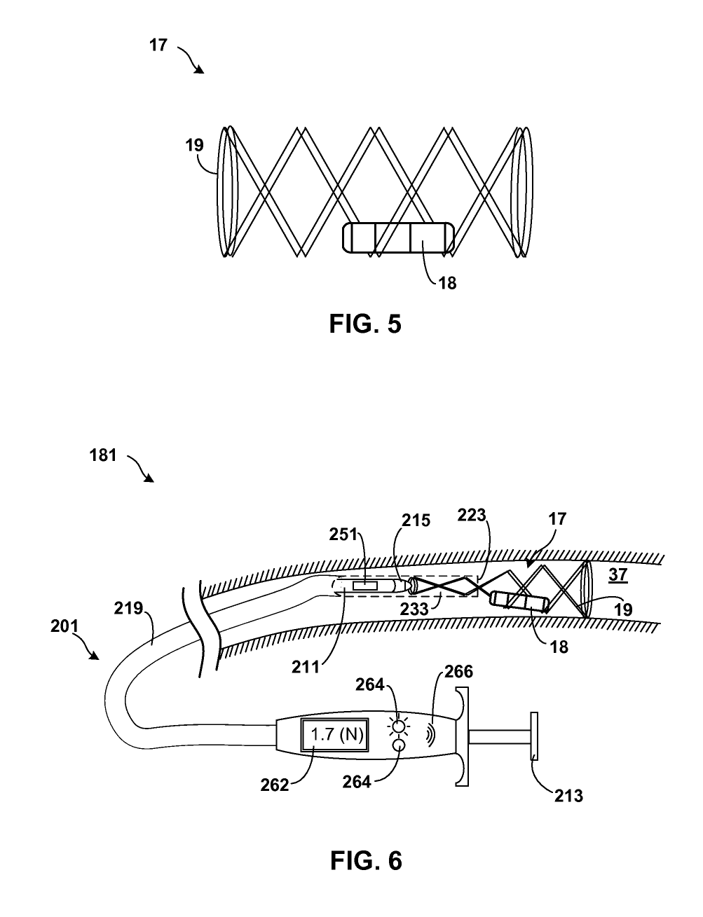

FIG. 5 illustrates leadless IMD 17 of FIG. 2 in further detail. In the example of FIG. 5, leadless IMD 17 includes expandable fixation element 19 and electronic subassembly 18. Electronic subassembly 18 includes control electronics that control the sensing and/or therapy functions of IMD 17 and a battery that powers the control electronics. As one example, the control electronics may include sensing circuitry and a telemetry module. Moreover, the battery may comprise features of the batteries disclosed in U.S. patent application Ser. No. 12/696,890, titled IMPLANTABLE MEDICAL DEVICE BATTERY and filed Jan. 29, 2010, the contents of which were previously incorporated by reference herein. The housing of electronic subassembly 18 may be formed from a biocompatible material, such as stainless steel and/or titanium alloys.

Expandable fixation element 19 is attached to electronic subassembly 18 and configured to anchor leadless IMD 17 within pulmonary artery 37, or other body lumen such as another vasculature. In particular, expandable fixation element 19 is deployable from a collapsed position to an expanded position such that outer diameter of expandable fixation element 19 provides an interference fit with the inner diameter of pulmonary artery 37, or other body lumen. Expandable fixation element 19 is shown in an expanded position in FIG. 5.

Expandable fixation element 19 may be fabricated of a shape memory material that allows expandable fixation element 19 to bend elastically from the collapsed position to the expanded position. As an example, the shape memory material may be shape memory alloy such as Nitinol. As an example, expandable fixation element 19 may store less potential energy in the expanded position and thus be naturally biased to assume the expanded position when in the collapsed position. In this manner, expandable fixation element 19 may assume an expanded position when no longer constrained by a catheter or other delivery device.

In some examples, expandable fixation element 19 may resemble a stent. Techniques for a partially deployable stents that may be applied to expandable fixation element 19 are disclosed in U.S. Pat. Pub. No. 2007/0043424, titled, “RECAPTURABLE STENT WITH MINIMUM CROSSING PROFILE” and dated Feb. 22, 2007, the entire contents of which are incorporated by reference herein, as well as U.S. Pat. Pub. No. 2009/0192585, titled, “DELIVERY SYSTEMS AND METHODS OF IMPLANTATION FOR PROSTETIC HEART VALVES” and dated Jul. 30, 2009, the entire contents of which are also incorporated by reference herein.

In some examples, all or a portion of expandable fixation element 19, such as active fixation tines 103, may include one or more coatings. For example, fixation element 102 may include a radiopaque coating to provide visibility during fluoroscopy. As another example, expandable fixation element 19 may be coated with a tissue growth promoter or a tissue growth inhibitor.

FIG. 6 illustrates assembly 181, which includes leadless IMD 17 and catheter 201. Catheter 201 is configured to deliver leadless IMD 17 to a pulmonary artery 37 or another location, e.g., within the vasculature, of a patient and remotely deploy IMD 17. Catheter 201 may be a steerable catheter or be configured to traverse a guidewire and may be directed within a body lumen, such as a vascular structure to a target site in order to facilitate remote positioning and deployment of IMD 17. FIG. 6 illustrates expandable fixation element 19 of IMD deployed in pulmonary artery 37.

Catheter 201 comprises outer member 219 and inner member 211. Outer member 219 forms lumen 233, which is sized to receive IMD 17 at distal end 223 of catheter 201 when IMD 17 is in a collapsed position. For example, the inner diameter of lumen 233 may be about the same size as the outer diameter of IMD 17 when IMD 17 is in a collapsed position. When IMD 17 is positioned within lumen 233 at the distal end of catheter 201, lumen 233 of outer member 219 constrains IMD 17 and holds expandable fixation element 19 in a collapsed position. As expandable fixation element 19 may be biased towards an expanded position, expandable fixation element 19 may assume a collapsed position with a diameter about equal to inner diameter of lumen 233 even if expandable fixation element 19 could potentially collapse to a diameter smaller than the inner diameter of lumen 233.

Inner member 211 is positioned proximate to IMD 17 in lumen 233 Inner member 211 configured to initiate deployment of IMD 17. More particularly, a clinician may remotely deploy IMD 17 by pressing plunger 213, which is located at the proximal end of catheter 201. Plunger 213 connects directly to inner member 211, e.g., with a wire or other stiff element running through catheter 201, such that pressing on plunger 213 moves inner member 211 distally within lumen 233. As inner member 211 moves distally within lumen 233, inner member 211 pushes IMD 17 distally within lumen 233. Inner member 211 also include release mechanism 215, which can be used to selectively release the proximal end of IMD 17 from catheter 201. In one example, release mechanism 215 can consist of a looped suture that is selectively released with a pull wire that is in mechanical communication with the proximal end of the catheter 201. Exemplary techniques suitable for release mechanism 215 are disclosed by U.S. Pat. No. 6,350,278, titled APPARATUS AND METHODS FOR PLACEMENT AND REPOSITIONING OF INTRALUMINAL PROSTHESES and issued Feb. 26, 2002, the entire contents of which are incorporated by reference herein.

As shown in FIG. 6, expandable fixation element 19 is partially deployable. The distal end of expandable fixation element 19 is in an expanded position and provides an interference fit with pulmonary artery 37, while the proximal end of expandable fixation element 19 remains in a collapsed position within distal end 223 of catheter 201. To prevent accidental full deployment of expandable fixation element 19 plunger 213 may include a positive stop prior to pushing expandable fixation element 19 completely out of lumen 233. As another example, plunger 213 may move far enough to push expandable fixation element 19 completely out of lumen 233. In such an example, full deployment of IMD 17 would require withdrawing catheter 201 while actuating release mechanism 215.

The expandable fixation element 19 allows IMD 17 to be retracted before fully deploying IMD 17, e.g., to adjust the position of IMD 17 with a vasculature to provide a tighter (or looser) interference fit. For example, a clinician implanting IMD 17 may reposition IMD 17 during an implantation procedure if partial deployment of the expandable fixation element provides an insufficient holding force indicating that full deployment of the expandable fixation element may not reliably secure IMD 17 within the vasculature. As another example, a clinician may select a different expandable fixation element with a different size that is better suited for a selected vasculature position.

Following partial deployment of IMD 17, a clinician may remotely pull IMD 17 back into lumen 233 by pulling plunger 213. Pulling IMD 17 back into lumen 233 returns expandable fixation element 19 to the collapsed position from the expanded position. A clinician may redeploy IMD 17 with inner member 211 by operating plunger 213.

Catheter 201 further includes force sensor 251, which is located on inner member 211. Force sensor 251 is in mechanical communication with IMD 17 via inner member 211. Force sensor 251 collects force feedback data representing force applied by inner member 211 on IMD 17. For example, force sensor 251 collects force feedback data representing both pull and push forces of inner member 211 on IMD 17. Force sensor 251 is located near the distal end of inner member 211 so that measurements are not significantly impacted by friction between outer member 219 and inner member 211.

In different examples, force sensor 251 may be a fiber optic force sensor or a strain gauge, such as a quarter bridge strain gauge. Strain gauges suitable for use as force sensor 251 include the Arthroscopically Implantable Force Probe that is available from MicroStrain, Inc. of Williston Vt., United States of America.

Force sensor 251 may be used by a clinician to determine if a holding force of IMD 17 at least meets a predetermined threshold level, e.g., a holding force associated with an acceptably low risk of future migration or dislodgement of IMD 17. To determine whether a holding force of IMD 17 at least meets a predetermined threshold level, a clinician first partially deploys expandable fixation element 19 into pulmonary artery 37 such that at least the distal end of expandable fixation element 19 is in an expanded position to create an interference fit with the inner diameter of pulmonary artery 37. Then the clinician pulls on inner member 211 at the proximal end of catheter 201 while monitoring force feedback information corresponding to the force feedback data collected by force sensor 251. Once the force feedback information monitored by the clinician indicates that the holding force of IMD 17 at least meets a predetermined threshold level, the clinician may stop pulling on inner member 211 to prevent dislodging IMD 17 from pulmonary artery 37. Alternatively, if the holding force of IMD 17 does not at least meet a predetermined threshold level, IMD 17 may migrate within or dislodge from pulmonary artery 37 before the force feedback information indicates that the holding force of IMD 17 at least meets a predetermined threshold level. In one example, a clinician may monitor the position of IMD 17 using fluoroscopy while pulling on inner member 211 to detect migration of IMD 17.

In another example, force sensor 251 further collects force feedback information representing a pushing force of inner member 211 on IMD 17 as a clinician user attempts to deploy IMD 17 from catheter 201. Such force information could indicate to a clinician a potential a hang-up between IMD 17 and catheter 201, e.g., between expandable fixation element 19 and an inner wall of outer member 219 or more importantly, excessive deployment force being applied on patient tissue during deployment, which could cause injury to the patient tissue, such as a rupturing a vasculature. In such an instance, the clinician could readjust positioning of catheter 201 and reattempt deployment rather than risk injury to the patient tissue.

Catheter 201 includes a variety of exemplary user communication modules suitable for delivering force feedback information corresponding to the force feedback data collected by force sensor 251 to the clinician. For example, as discussed with respect to catheter 200, catheter 201 may include digital readout 262, visible alert 264, and audible alert 266. In other examples, a clinician may receive force feedback information corresponding to the force feedback data collected by force sensor 251 from a device, e.g., a device similar to programmer 24, that is in wireless communication with force sensor 251.

Based on the force feedback information collected by force sensor 251, the clinician can to determine if the holding force of IMD 17 is adequate before fully releasing IMD 17 from catheter 201. Fully releasing IMD 17 from the catheter 201 includes releasing IMD 17 from inner member 211 by actuating release mechanism 215 using a control on the proximal end of catheter 201 (not shown) and withdrawing catheter 201 such that the entirety of IMD 17 exits lumen 233 at distal end 223 of catheter 201.

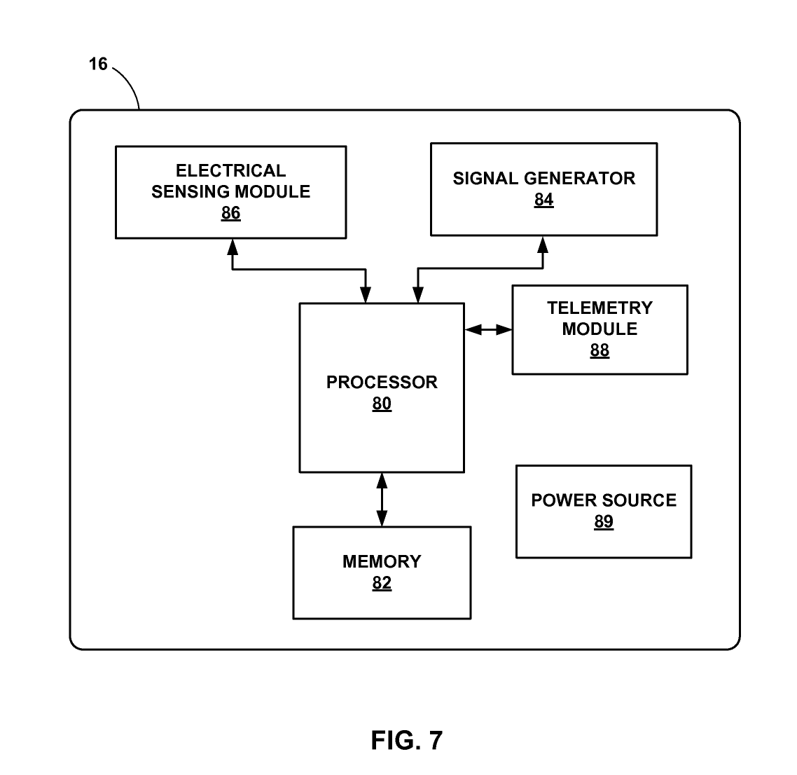

FIG. 7 is a functional block diagram illustrating one example configuration of IMD 16 of FIG. 1. In the example illustrated by FIG. 7, IMD 16 includes a processor 80, memory 82, signal generator 84, electrical sensing module 86, telemetry module 88, and power source 89. Memory 82 may include computer-readable instructions that, when executed by processor 80, cause IMD 16 and processor 80 to perform various functions attributed to IMD 16 and processor 80 herein. Memory 82 may be a computer-readable storage medium, including any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 in this disclosure may be embodied as software, firmware, hardware or any combination thereof. Processor 80 controls signal generator 84 to deliver stimulation therapy to heart 12 according to operational parameters or programs, which may be stored in memory 82. For example, processor 80 may control signal generator 84 to deliver electrical pulses with the amplitudes, pulse widths, frequency, or electrode polarities specified by the selected one or more therapy programs.

Signal generator 84, as well as electrical sensing module 86, is electrically coupled to electrodes of IMD 16. In the example illustrated in FIG. 7, signal generator 84 is configured to generate and deliver electrical stimulation therapy to heart 12. For example, signal generator 84 may deliver pacing, cardioversion, defibrillation, and/or neurostimulation therapy via at least a subset of the available electrodes. In some examples, signal generator 84 delivers one or more of these types of stimulation in the form of electrical pulses. In other examples, signal generator 84 may deliver one or more of these types of stimulation in the form of other signals, such as sine waves, square waves, or other substantially continuous time signals.

Signal generator 84 may include a switch module and processor 80 may use the switch module to select, e.g., via a data/address bus, which of the available electrodes are used to deliver stimulation signals, e.g., pacing, cardioversion, defibrillation, and/or neurostimulation signals. The switch module may include a switch array, switch matrix, multiplexer, or any other type of switching device suitable to selectively couple a signal to selected electrodes.

Electrical sensing module 86 monitors signals from at least a subset of the available electrodes, e.g., to monitor electrical activity of heart 12. Electrical sensing module 86 may also include a switch module to select which of the available electrodes are used to sense the heart activity. In some examples, processor 80 may select the electrodes that function as sense electrodes, i.e., select the sensing configuration, via the switch module within electrical sensing module 86, e.g., by providing signals via a data/address bus.

In some examples, electrical sensing module 86 includes multiple detection channels, each of which may comprise an amplifier. Each sensing channel may detect electrical activity in respective chambers of heart 12 and may be configured to detect either R-waves or P-waves. In some examples, electrical sensing module 86 or processor 80 may include an analog-to-digital converter for digitizing the signal received from a sensing channel for electrogram (EGM) signal processing by processor 80. In response to the signals from processor 80, the switch module within electrical sensing module 86 may couple the outputs from the selected electrodes to one of the detection channels or the analog-to-digital converter.

During pacing, escape interval counters maintained by processor 80 may be reset upon sensing of R-waves and P-waves with respective detection channels of electrical sensing module 86. Signal generator 84 may include pacer output circuits that are coupled, e.g., selectively by a switching module, to any combination of the available electrodes appropriate for delivery of a bipolar or unipolar pacing pulse to one or more of the chambers of heart 12. Processor 80 may control signal generator 84 to deliver a pacing pulse to a chamber upon expiration of an escape interval. Processor 80 may reset the escape interval counters upon the generation of pacing pulses by signal generator 84, or detection of an intrinsic depolarization in a chamber, and thereby control the basic timing of cardiac pacing functions. The escape interval counters may include P-P, V-V, RV-LV, A-V, A-RV, or A-LV interval counters, as examples. The value of the count present in the escape interval counters when reset by sensed R-waves and P-waves may be used by processor 80 to measure the durations of R-R intervals, P-P intervals, P-R intervals and R-P intervals. Processor 80 may use the count in the interval counters to detect heart rate, such as an atrial rate or ventricular rate. In some examples, a leadless IMD with a set of active fixation tines may include one or more sensors in addition to electrical sensing module 86. For example, a leadless IMD may include a pressure sensor and/or a tissue oxygen sensor.

Telemetry module 88 includes any suitable hardware, firmware, software or any combination thereof for communicating with another device, such as programmer 24 (FIGS. 1 and 2). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and receive downlinked data from programmer 24 via an address/data bus. In some examples, telemetry module 88 may provide received data to processor 80 via a multiplexer.

In some examples, processor 80 may transmit an alert that a mechanical sensing channel has been activated to identify cardiac contractions to programmer 24 or another computing device via telemetry module 88 in response to a detected failure of an electrical sensing channel. The alert may include an indication of the type of failure and/or confirmation that the mechanical sensing channel is detecting cardiac contractions. The alert may include a visual indication on a user interface of programmer 24. Additionally or alternatively, the alert may include vibration and/or audible notification. Processor 80 may also transmit data associated with the detected failure of the electrical sensing channel, e.g., the time that the failure occurred, impedance data, and/or the inappropriate signal indicative of the detected failure.

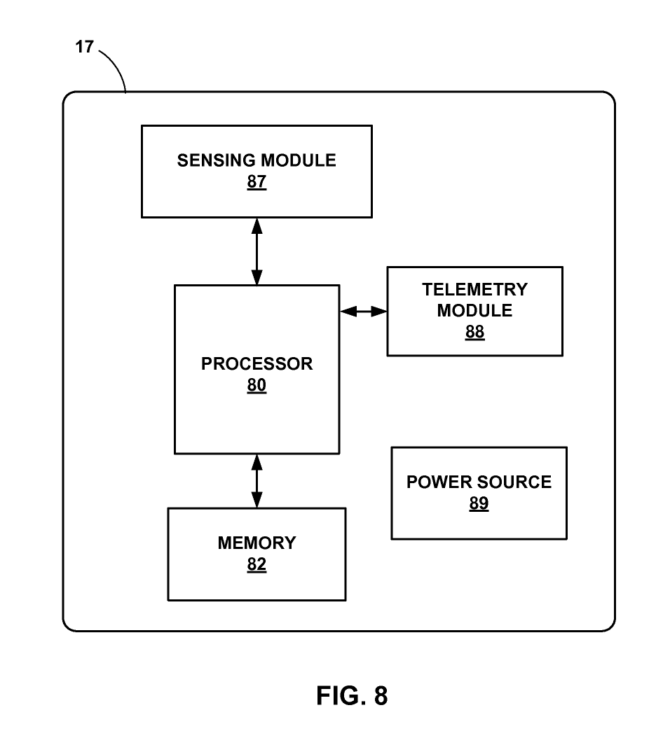

FIG. 8 is a functional block diagram illustrating one example configuration of IMD 17 of FIG. 2. In the example illustrated by FIG. 8, IMD 17 includes a processor 80, memory 82, sensing module 87, telemetry module 88, and power source 89. The functional block diagram of IMD 17 is substantially similar to the functional block diagram of IMD 16 shown in FIG. 6. One exception is that IMD 17 includes sensing module 87, but does not include signal generator 84 or electrical sensing module 86. For brevity, components discussed with respect to IMD 16 are not discussed with respect to IMD 17.

Sensing module 87 may include a pressure sensor, e.g., to measure pressure within a vasculature of a patient. Additionally or alternatively to including a pressure sensor, sensing module 87 may also include sensors such as, but not limited to an electrocardiogram sensor, a fluid flow sensor, an oxygen sensor (for tissue oxygen or blood oxygen sensing), an accelerometer, a glucose sensor, a potassium sensor, a thermometer and/or other sensors.

FIG. 9 is a functional block diagram of an example configuration of programmer 24. As shown in FIG. 9, programmer 24 includes processor 90, memory 92, user interface 94, telemetry module 96, and power source 98. Programmer 24 may be a dedicated hardware device with dedicated software for programming one of IMDs 16, 17. Alternatively, programmer 24 may be an off-the-shelf computing device running an application that enables programmer 24 to program IMDs 16, 17.

A user, such as a clinician, may use programmer 24 to select therapy programs (e.g., sets of stimulation parameters), generate new therapy programs, or modify therapy programs for IMDs 16, 17. The user may also use programmer 24 to select sensing parameters and/or retrieve patient data including but not limited to a therapy history and or sensor data associated with the IMD. The user may interact with programmer 24 via user interface 94, which may include a display to present a graphical user interface to a user, and a keypad or another mechanism for receiving input from a user.

Processor 90 can take the form of one or more microprocessors, DSPs, ASICs, FPGAs, programmable logic circuitry, or the like, and the functions attributed to processor 90 in this disclosure may be embodied as hardware, firmware, software or any combination thereof. Memory 92 may store instructions and information that cause processor 90 to provide the functionality ascribed to programmer 24 in this disclosure. Memory 92 may include any fixed or removable magnetic, optical, or electrical media, such as RAM, ROM, CD-ROM, hard or floppy magnetic disks, EEPROM, or the like. Memory 92 may also include a removable memory portion that may be used to provide memory updates or increases in memory capacities. A removable memory may also allow patient data to be easily transferred to another computing device, or to be removed before programmer 24 is used to program therapy for another patient. Memory 92 may also store information that controls therapy delivery by IMDs 16, 17, such as stimulation parameter values.

Programmer 24 may communicate wirelessly with IMDs 16, 17, such as using RF communication or proximal inductive interaction. This wireless communication is possible through the use of telemetry module 96, which may be coupled to an internal antenna or an external antenna. An external antenna that is coupled to programmer 24 may correspond to the programming head that may be placed over heart 12, as described above with reference to FIG. 1. Telemetry module 96 may be similar to telemetry module 88 of IMD 16 (FIG. 7).

Telemetry module 96 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth® specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMDs 16, 17.

In some examples, processor 90 of programmer 24 and/or one or more processors of one or more networked computers may perform all or a portion of the techniques described in this disclosure with respect to processor 80 and IMDs 16, 17. For example, processor 90 or another processor may receive one or more signals from electrical sensing module 86, sensing module 87, or information regarding sensed parameters from IMDs 16, 17 via telemetry module 96. In some examples, processor 90 may process or analyze sensed signals, as described in this disclosure with respect to IMDs 16, 17 and processor 80.

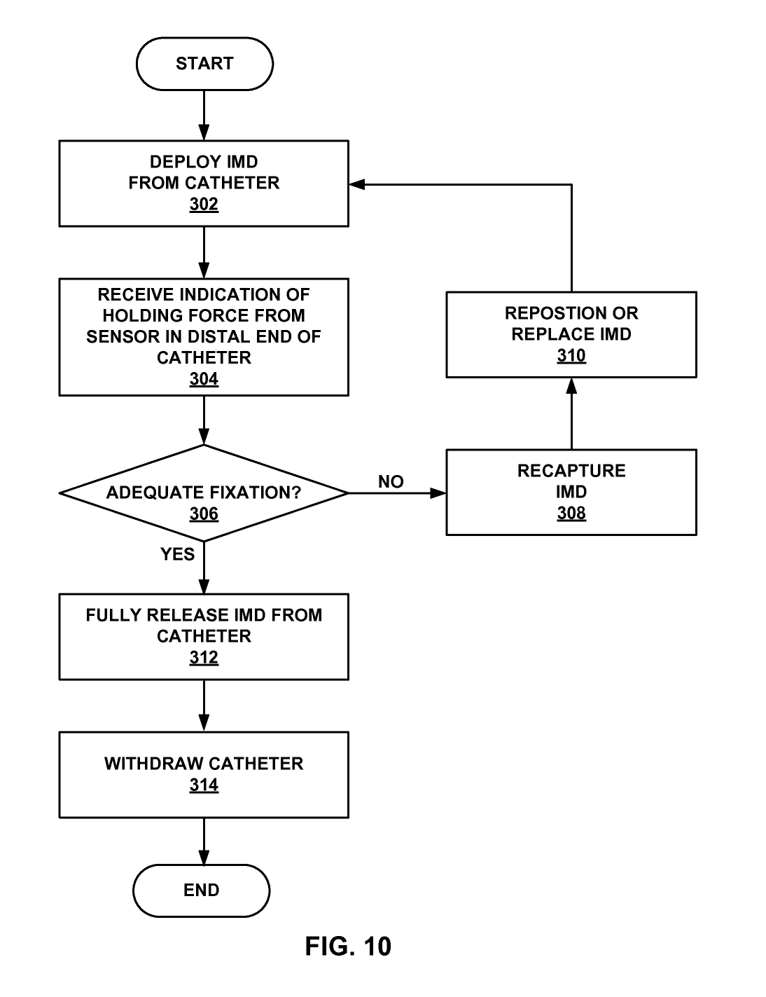

FIG. 10 is a flowchart illustrating techniques for implanting an implantable medical device within a patient. The techniques of FIG. 10 are described with respect to IMD 17, but are also applicable to IMD 16 as well as other IMDs.

First, IMD 17 is at least partially deployed from catheter 201 to a location within the patient, such as pulmonary artery 37, other vasculature of the patient, or a right ventricle of the patient (302). Catheter 201 includes force sensor 251 in mechanical communication with IMD 17. Next, a clinician receives an indication of a holding force of IMD 17. The indication of the holding force corresponds to force feedback data collected by force sensor 251 (304). For example, the clinician may pull on plunger 213 to applying an axial force to the deployed IMD 17 via a user-controlled portion of the catheter such as plunger 13, and the indication of the holding force of IMD 17 is a representation the axial force applied to the deployed IMD 17 via the user-controlled portion of the catheter. Fluroscope or other imaging, or a navigation technique to monitor location/motion, can be used by a physician at the same time the holding force of the IMD 17 is tested (304) in order to provide confirmation if IMD 17 has physically moved or dislodged prior to reaching holding force threshold.

The clinician evaluates whether IMD 17 is adequately fixated within the patient based on the indication of the holding force of IMD 17 (306). If the clinician determines IMD 17 is inadequately fixated within the patient, the clinician operates catheter 201 to recapture IMD 17 using inner member 211, e.g., by pulling on plunger 213 (308). Then, the clinician either repositions distal end 223 of catheter 201 or replaces IMD 17 with another IMD better sized for the implantation location (310). Then step 302 (see above) is repeated.

Once the clinician determines IMD 17 is adequately fixated within the patient based on the indication of the holding force of IMD 17 (306), the clinician operates catheter 201 to fully release IMD 17 within the patient, e.g., by actuating release mechanism 215 (312). Then, the clinician withdraws catheter 201, leaving IMD 17 secured within the patient (314).

Various examples of the disclosure have been described. These and other examples are within the scope of the following claims.

1. A kit for implanting an implantable medical device within a patient, the kit comprising:
a delivery catheter including an inner member and an outer member;
the implantable medical device, wherein the implantable medical device is adjacent the inner member and constrained by the outer member;
a force sensor in mechanical communication with the implantable medical device via the inner member, wherein the force sensor collects force feedback data representing force applied by the inner member on the implantable medical device; and
a user communication module configured to deliver force feedback information corresponding to the force feedback data collected by the force sensor to a user.

2. The kit of claim 1, wherein the implantable medical device is releasably attached to the inner member.

3. The kit of claim 1, wherein the force feedback information delivered to the user allows the user to evaluate whether the implantable medical device is adequately fixated within the patient prior to fully releasing the implantable medical device from the delivery catheter.

4. The kit of claim 1, wherein the force feedback information includes an indication that a holding force of the implantable medical device at least meets a predetermined threshold level.

5. The kit of claim 1, wherein the implantable medical device includes an expandable fixation mechanism deployable from a collapsed position to an expanded position, the expanded position suitable to secure the implantable medical device within a vascular structure of a patient.

6. The kit of claim 5, wherein the implantable medical device is configured for implantation within a pulmonary artery of the patient.

7. The kit of claim 1, wherein the implantable medical device includes a pressure sensor.

8. The kit of claim 1, wherein the implantable medical device includes an active fixation mechanism configured to secure the implantable medical device component to a patient tissue.

9. The kit of claim 8, wherein the active fixation mechanism includes a set of active fixation tines that are deployable from a spring-loaded position in which distal ends of the active fixation tines point away from a implantable medical device housing to a hooked position in which the active fixation tines bend back towards the implantable medical device housing.

10. The kit of claim 1, wherein the implantable medical device is a leadless pacemaker.

11. The kit of claim 1, wherein the force feedback information delivered to the user represents a pushing force of the inner member on the implantable medical device as the user attempts to deploy the implantable medical device from the catheter.

12. The kit of claim 1, wherein the force sensor includes at least one from a group consisting of:
a strain gauge; and
a fiber optic force sensor.

13. The kit of claim 1, wherein the implantable medical device includes at least one sensor selected from a group consisting of:
an electrocardiogram sensor;
a fluid flow sensor;
an oxygen sensor;
an accelerometer;
a glucose sensor;
a potassium sensor; and
a thermometer.

14. A catheter for implanting an implantable medical device within a patient, the catheter comprising:
an inner member configured to apply a force to the implantable medical device;
an outer member configured to constrain the implantable medical device;
a force sensor configured to collect force feedback data representing force applied by the inner member on the implantable medical device; and
a user communication module configured to deliver force feedback information corresponding to the force feedback data collected by the force sensor to a user.

15. The catheter of claim 14, wherein the inner member configured to releasably attach to the implantable medical device.

16. The catheter of claim 14, wherein the force feedback information delivered to the user allows the user to evaluate whether the implantable medical device is adequately fixated within a patient prior to fully releasing the implantable medical device from the inner member.

17. The catheter of claim 14, wherein the force feedback information includes an indication that a holding force of the implantable medical device at least meets a predetermined threshold level.

18. The catheter of claim 14,
wherein the inner member includes a tether and a deployment element,
wherein the force sensor is located on the tether.

19. The catheter of claim 14,
wherein the implantable medical device is configured for implantation to a location within the patient selected from a group consisting of:
a pulmonary artery of the patient; and
a right ventricle of the patient,
wherein the catheter is configured to deliver the implantable medical device to the location.

20. The catheter of claim 14, wherein the force feedback information delivered to the user represents a pushing force of the inner member on the implantable medical device as the user attempts to deploy the implantable medical device from the catheter.

21. The catheter of claim 14, wherein the force sensor includes at least one from a group consisting of:
a strain gauge; and
a fiber optic force sensor.

22. The catheter of claim 14, wherein the user communication module includes at least one from a group consisting of:
an audible alert;
a visible alert;
a digital readout providing a real-time representation of the force feedback data; and
a graphical user interface display of force versus time.

23. A method of implanting an implantable medical device within a patient comprising:
deploying the implantable medical device from a catheter to a location within the patient, the catheter including a force sensor in mechanical communication with the implantable medical device;
receiving an indication of a holding force of the implantable medical device, wherein the indication of the holding force corresponds to force feedback data collected by the force sensor; and
fully releasing the implantable medical device from the catheter at the location within the patient after determining the implantable medical device is adequately fixated at the location within the patient,
wherein determining the implantable medical device is adequately fixated at the location within the patient comprises evaluating whether the implantable medical device is adequately fixated at the location within the patient based on the indication of the holding force of the implantable medical device.

24. The method of claim 23, further comprising:
determining the implantable medical device is inadequately fixated within the patient; and
after determining the implantable medical device is inadequately fixated within the patient, recapturing the implantable medical device using the catheter prior to fully releasing the implantable medical device within the patient.

25. The method of claim 23, further comprising:
applying an axial force to the deployed implantable medical device via a user-controlled portion of the catheter,
wherein the indication of the holding force of the implantable medical device is a representation of the axial force applied to the deployed implantable medical device via the user-controlled portion of the catheter.

26. The method of claim 23, wherein the location is within a vasculature of the patient.

27. The method of claim 23, wherein the location is within a pulmonary artery of the patient.

28. The method of claim 23, wherein the location is within a right ventricle of the patient,

29. The method of claim 23, wherein the implantable medical device is a leadless pacemaker.
